(19) United States
(12) Patent Application Publication
(10) Pub. No.: US 2025/0127560 A1
(43) Pub. Date: Apr. 24, 2025

(54) PERCUTANEOUS VASCULAR ACCESS WITH RADIOFREQUENCY ENERGY

(71) Applicant: Boston Scientific Scimed, Inc., Maple Grove, MN (US)

(72) Inventors: Allan Charles Shuros, St. Paul, MN (US); Eduardo Moriyama, Richmond (CA)

(21) Appl. No.: 18/914,986

(22) Filed: Oct. 14, 2024

Related U.S. Application Data
(60) Provisional application No. 63/591,726, filed on Oct. 19, 2023.

(51) Int. Cl. A61B 18/14; A61B 18/00; A61B 18/12

(52) U.S. Cl. CPC A61B 18/1487; A61B 18/1206; A61B 2018/00184; A61B 2018/00351; A61B 2018/00595; A61B 2018/00636; A61B 2018/00702; A61B 2018/1253; A61B 2018/144; A61B 2018/1475

(57) ABSTRACT

A percutaneous vascular access assembly for use with a radiofrequency (RF) energy source is disclosed. The percutaneous vascular access assembly includes an RF perforation instrument to be coupled to the RF energy source. The RF perforation instrument has an elongate shaft defining a longitudinally extending lumen and an instrument distal tip having an instrument electrode to deliver the RF energy. A puncture member is to be disposed within the lumen and extendable from the instrument distal tip. The puncture member is to be coupled to the RF energy source. The puncture member includes a puncture member distal tip having a puncture member electrode to deliver the RF energy.

### CROSS REFERENCE TO RELATED APPLICATIONS

This application claims priority to U.S. Provisional Patent Application No. 63/591,726 entitled, “PERCUTANEOUS VASCULAR ACCESS WITH RADIOFREQUENCY ENERGY,” filed Oct. 19, 2024, which is incorporated herein by reference in its entirety.

### TECHNICAL FIELD

The present disclosure relates to medical devices and systems for use in percutaneous or interventional procedures including surgery. More specifically, this disclosure relates to electrosurgical devices, assemblies, systems, and methods that provide for percutaneous vascular access via radiofrequency (RF) energy provided to an electrode.

### BACKGROUND

Some medical procedures provide intervention at a desired target tissue site within a region of tissue within a patient's body. For example, a medical procedure provides access to a particular cavity or space such as a chamber of the heart or the pericardium of the heart to provide treatment to the heart via cardiac catheterization. In some applications, access to the cavity or space may be provided through a puncture that is created within the desired tissue site. To initially reach the desired site within the region of tissue, the procedure can provide percutaneous access to a patient's blood vessel, and then access to the tissue site is provided into through vasculature using a guidewire. A sheath and dilator assembly may then be advanced over the guide wire, and the sheath may be used to guide the dilator, as well as any other devices positioned through the assembly, to the desired target tissue site.

### SUMMARY

In an Example 1, a percutaneous vascular access assembly for use with a radiofrequency (RF) energy source, the percutaneous vascular access assembly comprising: an RF perforation instrument configured to be coupled to the RF energy source, the RF perforation instrument having an elongate shaft defining a longitudinally extending lumen and an instrument distal tip having an instrument electrode configured to deliver the RF energy; and a puncture member adapted to be disposed within the lumen and extendable from the instrument distal tip and configured to be coupled to the RF energy source, the puncture member having a puncture member distal tip having a puncture member electrode configured to deliver the RF energy.

In an Example 2, the percutaneous vascular access assembly of Example 1, and further comprising a cannula configured to receive the RF perforation instrument.

In an Example 3, the percutaneous vascular access assembly of any of Examples 1 and 2, wherein the RF perforation instrument is configured as a trocar.

In an Example 4, the percutaneous vascular access assembly of any of Examples 1-3, wherein the RF perforation instrument includes a rigid conductive shaft covered in an electrical insulative material that exposes the instrument electrode.

In an Example 5, the percutaneous vascular access assembly of any of Examples 1-4, wherein the instrument electrode is an exposed distal-facing tip of the elongate shaft.

In an Example 6, the percutaneous vascular access assembly of any of Examples 1-5, wherein the RF perforation instrument includes a sensor.

In an Example 7, the percutaneous vascular access assembly of Example 6, wherein the sensor includes at least one of an impedance sensor, temperature sensor, and pressure sensor.

In an Example 8, the percutaneous vascular access assembly of any of Examples 1-6, wherein the puncture member is configured as a multifunction guidewire.

In an Example 9, the percutaneous vascular access assembly of Example 8, wherein the puncture member distal tip is extendable from the instrument distal tip such that the RF perforation instrument is fully retractable over the puncture member.

In an Example 10, the percutaneous vascular access assembly of any of Examples 8-9, wherein the guidewire includes an atraumatic distal section.

In an Example 11, the percutaneous vascular access assembly of any of Examples 1-10, and further comprising an RF generator to provide the RF energy source.

In an Example 12, the percutaneous vascular access assembly of any of Examples 1-11, wherein the RF perforation instrument and the puncture member are configured to deliver the RF energy in a monopolar mode.

In an Example 13, the percutaneous vascular access assembly of any of Examples 1-12, wherein the RF perforation instrument is configured to advance against tissue in the body, the distal tip configured to puncture tissue at a tissue site, and wherein the puncture member is configured to advance against a vessel in the body accessed from the puncture tissue, the guidewire electrode configured to puncture the vessel at the puncture site.

In an Example 14, the percutaneous vascular access assembly of Example 13, wherein the RF perforation instrument punctures the tissue with a first RF energy and the puncture member punctures the vessel with a second RF energy, the first RF energy different than the second RF energy.

In an Example 15, the percutaneous vascular access assembly of Example 13, wherein the puncture member cauterizes the puncture site with a third RF energy, wherein the third RF energy is different than the second RF energy.

In an Example 16, a method of percutaneously accessing vasculature in a body, the method comprising: advancing, against tissue in the body, a percutaneous instrument having an elongate shaft defining a longitudinal lumen and an instrument distal tip, the instrument distal tip configured to puncture the tissue at a puncture site; and advancing, against a vessel in the body accessed from the punctured tissue, an RF perforation guidewire extendable from the lumen, the RF perforation guidewire having an atraumatic guidewire distal tip, the guidewire distal tip having a guidewire electrode delivering RF energy to puncture the vessel at the puncture site.

In an Example 17, the method of Example 16, wherein the instrument distal tip includes a needle mechanically puncturing the tissue at the puncture site.

In an Example 18, the method of Example 16, wherein the instrument distal tip includes an instrument electrode configured to deliver RF energy to puncture the tissue at the puncture site.

In an Example 19, the method of Example 18, wherein delivering the RF energy to puncture the tissue and delivering the RF energy to puncture the vessel include delivering the RF energy in a monopolar mode.

In an Example 20, the method of Example 16, wherein the RF perforation guidewire is introduced into the lumen after the vessel is punctured.

In an Example 21, the method of Example 16, wherein the guidewire distal tip is extendable from the instrument distal tip such that the percutaneous instrument is fully retractable over the RF perforation guidewire.

In an Example 22, the method of Example 16, further comprising advancing, into a left atrium of a heart of the body, the Rf perforation guidewire via the vessel from the puncture site.

In an Example 23, the method of Example 22, further comprising puncturing an atrial septum of the heart with the atraumatic guidewire distal tip having the guidewire electrode delivering RF energy.

In an Example 24, the method of Example 16, further comprising cauterizing the puncture site.

In an Example 25, a method of percutaneously accessing vasculature in a body, the method comprising: puncturing tissue at a puncture site in the body; puncturing a vessel in the body accessed from the punctured tissue, the puncturing the vessel with an electrode delivering a first RF energy at the puncture site; and cauterizing the puncture site with the electrode delivering a second RF energy, wherein the first RF energy is different from the second RF energy.

In an Example 26, the method of Example 25, wherein the puncturing the tissue includes puncture the tissue with the electrode delivering a third RF energy at the puncture site.

In an Example 27, the method of Example 26, wherein the first RF energy is the same as the third RF energy.

In an Example 28, a percutaneous vascular access assembly for use with a radiofrequency (RF) energy source, the percutaneous vascular access assembly comprising: an RF perforation instrument configured to be coupled to the RF energy source, the RF perforation instrument having an elongate shaft defining a longitudinally extending lumen and an instrument distal tip having an instrument electrode configured to deliver the RF energy; and a puncture member adapted to be disposed within the lumen and extendable from the instrument distal tip and configured to be coupled to the RF energy source, the puncture member having a puncture member distal tip having a puncture member electrode configured to deliver the RF energy.

In an Example 29, the percutaneous vascular access assembly of Example 28, and further comprising a cannula configured to receive the RF perforation instrument.

In an Example 30, the percutaneous vascular access assembly of Example 28, wherein the RF perforation instrument is configured as a trocar.

In an Example 31, the percutaneous vascular access assembly of Example 28, wherein the RF perforation instrument includes a sensor.

In an Example 32, the percutaneous vascular access assembly of Example 28, wherein the puncture member is configured as a multifunction guidewire.

In an Example 33, the percutaneous vascular access assembly of Example 32, wherein the guidewire includes an atraumatic distal section.

In an Example 34, the percutaneous vascular access assembly of Example 28, and further comprising an RF generator to provide the RF energy source.

In an Example 35, the percutaneous vascular access assembly of Example 28, wherein the RF perforation instrument and the puncture member are configured to deliver the RF energy in a monopolar mode.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram illustrating an exemplary electrosurgical system for treating a patient, such as a heart or the vasculature of a patient including an inset illustrating a schematic diagram of a transseptal puncture performed with the example electrosurgical system.

FIG. 2 is a schematic diagram illustrating an embodiment of a vascular access assembly for use in the example electrosurgical system of FIG. 1.

FIG. 3 is a schematic diagram illustrating another embodiment of a vascular access assembly for use in the example electrosurgical system of FIG. 1.

FIG. 4 is a schematic diagram illustrating still another embodiment of a vascular access assembly for use in the example electrosurgical system of FIG. 1.

FIGS. 5A-5E are schematic diagrams illustrating an example process of using vascular access assemblies with the example electrosurgical system of FIG. 1.

FIGS. 6A-6E are schematic diagrams illustrating an example process of using vascular access assembly of FIG. 3.

FIG. 7 is a schematic diagram illustrating an example feature for use with the example processes of FIGS. 5 and 6.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

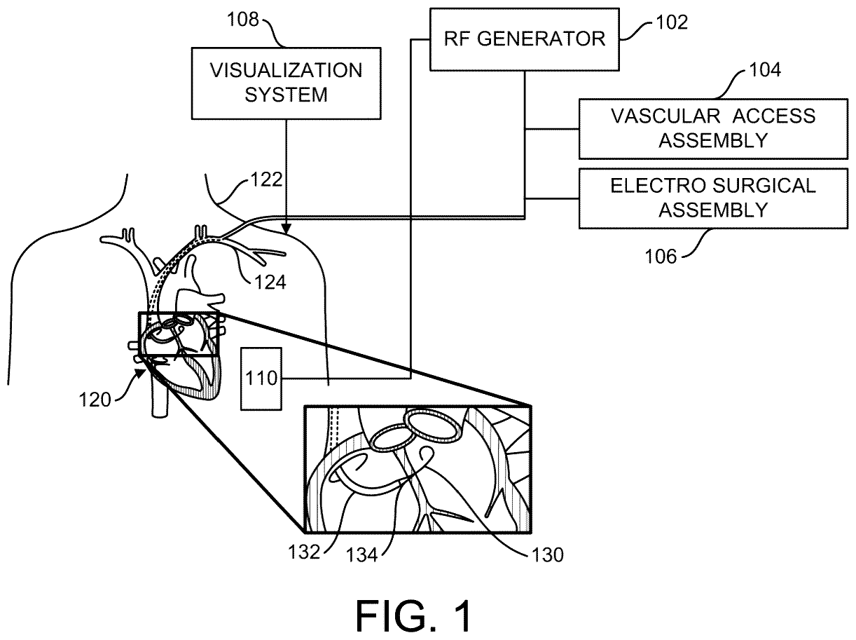

FIG. 1 illustrates a clinical setting having an embodiment of an electrosurgical medical system 100 to facilitate vascular access to a heart 120 and provide catheter positioning within cardiac anatomy. The medical system 100 includes an electrosurgical generator 102, a vascular access assembly 104, and electrosurgical assembly 106, and a visualization system 108. The electrosurgical generator 102 is electrically coupled to the vascular access assembly 104 and the electrosurgical assembly 106 to provide a source of energy, such as radiofrequency (RF) energy via the cables. In some embodiments, the electrosurgical medical system 100 includes a ground pad electrode, or indifferent (dispersive) patch electrode 110 electrically coupled to the electrosurgical generator 102 for use with the vascular access assembly 104 or electrosurgical assembly 106 in a monopolar configuration. In some embodiments, vascular access assembly 104 and electrosurgical assembly 106 are implemented in a bipolar configuration without an indifferent patch electrode. The visualization system 108, such as a fluoroscopic visualization system employing a C-arm, an echocardiogram system such as an intracardiac echocardiography (ICE) system a transesophageal electrocardiography (TEE) system, or an electroanatomical mapping (EAM) system can be implemented to track the locations of components of the electrosurgical system 106 and, in some instances, the vascular access system 104, within the body and with respect to structures and tissues within the body.

The RF energy is provided to the vascular access assembly 104 to percutaneously access the vasculature of a patient for catheters or components of the electrosurgical assembly 106. For instance, components of the vascular access assembly 104 are disposed against a patient's derma 122 at a puncture site, and the electrosurgical generator 102 is energized to puncture the tissue via RF perforation. Once a component of the vascular access assembly 104 is within a vessel, such as the femoral artery of the patient, components of the electrosurgical assembly 106 can be introduced into the vasculature 124. The components of the electrosurgical assembly 106 can be introduced into the vasculature 124 at the puncture site and navigated to the patient's heart 120, such as a chamber in the patient's heart 120.

The electrosurgical generator 102 is configured to provide the source of RF energy to the vascular access assembly 104 and the electrosurgical assembly 106 for a surgical procedure. The electrosurgical generator 102 is electrically coupled via cables to components of the vascular access assembly 104 and the electrosurgical assembly 106 and the patch electrode 110. During a monopolar operation of electrosurgical generator 102, a first electrode, often referred to as the active electrode, is provided with one of the vascular access assembly 104 and the electrosurgical assembly 106 while a second electrode, such as patch electrode 110, is typically located on the back, buttocks, upper leg, or other suitable anatomical location of the patient during surgery. In such a configuration, the patch electrode 110 is often referred to as a patient return electrode. In some embodiments, the vascular access assembly 104 or electrosurgical assembly 106 is implemented in a bipolar configuration without an indifferent patch electrode. An electrical circuit of RF energy is formed between the active electrode and the patch electrode 110 through the patient, which is used to puncture tissue at the active electrode. For example, RF energy for a puncture function in a monopolar mode may be provided at a relatively low voltage and a continuous current (100% on, or 100% duty cycle). Nominal impedance can range between 300 to 1000 ohms for the cutting function. At a power setting of 90 Watts for cutting, voltage can range from approximately 164 to 300 volts root mean square (RMS). The electrosurgical generator 102 can include a plurality of functions and provide a programmed and custom settings via an interface and be couplable to a suite of electrosurgical tools in addition components of the vascular access assembly 104 and the electrosurgical assembly 106. For instance, the electrosurgical generator 102 can include a first RF setting for a cut or puncture function, a second RF energy setting for a cauterization function, and another RF setting for a hemostasis setting, which is often implemented in a bipolar mode. Additionally, the electrosurgical generator 102 can include a plurality of setting for the functions.

The vascular access assembly 104 includes components couplable to the source of RF energy. In some embodiments, the vascular access assembly 104 includes a perforation instrument to puncture tissue, such as a trocar. The perforation instrument punctures tissue mechanically with a blade or via RF energy as an RF perforation instrument. In some embodiments, the RF perforation instrument is configured to also puncture a vessel. The perforation instrument can be inserted into a delivery device such as a cannula or sheath. In some embodiments, the delivery device can extend into the tissue to the vessel and, in some embodiments, puncture the vessel for vascular access. Some embodiments include a puncture member, such as a puncture guidewire or crossing member. The puncture member is delivered into the vasculature via the delivery device or from within the perforation instrument, such as within a lumen in the perforation instrument. In some embodiments, the perforation instrument is removed from the delivery device prior to insertion of the puncture member. The puncture member in some embodiments is coupled to the source the of RF energy to puncture the vessel.

Components of the visualization system 108 can include a device to receive tracking information such as transducers and field generators, a controller to process the tracking information and generate a visualization such as a processor with memory storing a computer program, and a display device to provide the visualization. The visualization system 108 in embodiments utilizes location information for the various tracked components, and at times along with cardiac electrical activity acquired by, for example, another catheter or probe equipped with sensing electrodes, to generate, and display via a display device, detailed multi-dimensional maps or representations of the heart tissue and voids such as cardiac chambers as well as maps in which cardiac electrical activity of interest is superimposed on the geometric anatomical maps. Furthermore, the visualization system 108 can generate a graphical representation of the various tracked devices within the maps on the display device. Components of the vascular access assembly 104, the electrosurgical assembly 106, or both can be equipped with electrodes, or radiopaque or echogenic markers to aid in visualization and identification of parameters such as spacings and relative locations.

The inset to FIG. 1 illustrates use of components of the electrosurgical assembly 106 applied in an example transeptal puncture of a cardiac procedure. The electrosurgical assembly 106 is implemented to create a channel through the interatrial septum from the right atrial to the left atrium of the heart 120. The left atrium is a difficult cardiac chamber to access reach percutaneously. Although the left atrium can be reached via the left ventricle and mitral valve with a catheter, the catheter is manipulated through two U-turns, which can be cumbersome. The transseptal puncture is a technique of creating a small surgical passage through the atrial septum, or wall in the heart between the left and right atrium, through which a catheter can be fed. For example, the atrial septum is punctured and dilated via components of the electrosurgical assembly 106. In the example, the components can include an electrified crossing member 130 electrically coupled to the RF generator 102 to puncture the atrial septum via RF perforation, the crossing member 130 carried within a sheath and dilator assembly 132 that has been navigated via the patient's vasculature to the right atrium. Transseptal punctures can be performed with the aid of a crossing member 130 in the form of a multifunction guidewire having electrodes energized with the RF generator 102 in a manner like other electrosurgical devices. The transseptal puncture permits a direct route to the left atrium via the intra-atrial septum and systematic venous system. The atrial septum is punctured with the crossing member 130 and the resulting opening is enlarged via a dilator tip 134 of the sheath and dilator assembly 132. Increasing larger and complex medical devices can be passed into the right atrium via the sheath of the sheath and dilator assembly 132. The increased interest in catheter perforation and its application in many other procedures has meant the transseptal puncture is a routine technique for interventional cardiologists and cardiac electrophysiologists.

Vascular access can provide challenges. For example, vascular access complications are often the most common adverse events in cardiac catheterization interventional procedures including pseudoaneurysms, hematomas, arteriovenous fistulas, dissections, and lacerations. In some such applications, a particular access point into the patient's vasculature may be dictated by, for example, treatment requirements or anatomical considerations. For example, patients with occluded or stenosed vasculature can require an alternate access point or particular attention to vascular access. Thus, in certain procedures, a particular tissue puncture site is preferred while the access point into the vasculature is also restricted. In some such procedures, such as procedures involving transseptal punctures, delivering treatment tools and assemblies from the access point to the tissue puncture site is difficult or involve many device exchanges.

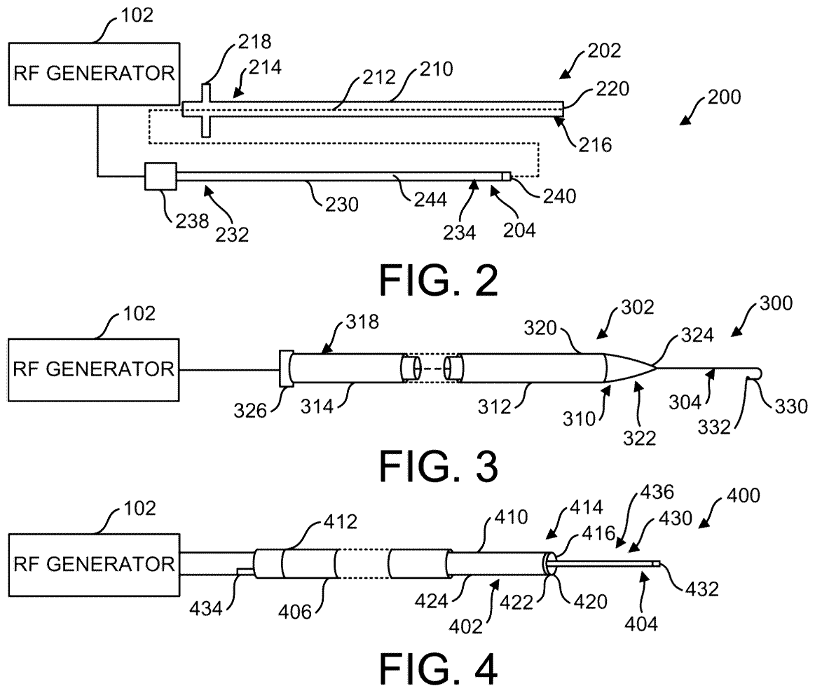

FIG. 2 illustrates an embodiment of a vascular access assembly 200 for use with electrosurgical medical system 100 as vascular assembly 104. The vascular access assembly 200 includes a delivery device such as a cannula 202 and a perforation instrument such as a trocar 204. In embodiments, the trocar 204 is configured to be coupled to the RF generator 102 of medical system 100 to deliver a source of RF energy.

The cannula 202 includes an elongate shaft 210 defining a longitudinally extending axial lumen 212. The shaft 210 includes a proximal end 214 and a distal end 216. An adapter 218 is coupled to the proximal end 214 in the illustrated embodiment. The distal end 216 of the cannula 202 includes a blunt tip 220. In some embodiments, the distal end 216 of the cannula 202 includes markers such as radiopaque or echogenic markers to enhance visualization and precise placement. The cannula shaft 210 is suitably rigid to provide for introduction into the patient and includes a suitable size to receive the trocar 204 within the cannula lumen 212.

The trocar 204 includes an elongate trocar shaft 230 having a proximal end 232 and a distal end 234. A trocar adapter 238 is coupled to the proximal end 232 of the trocar 204 in the illustrated embodiment. In some embodiments, the trocar adapter 238 and the cannula adapter 218 are configured to removably mate with each other, such as via male and female portions or via threaded portions. The distal end 234 of the trocar 204 includes a distal tip 240 configured to puncture tissue. In the illustrated embodiment of trocar 204, the distal tip 240 includes a trocar electrode 242 to deliver RF energy to puncture tissue. For instance, the trocar electrode 242 can be electrically coupled to the RF generator via elongate conductors disposed in the trocar shaft 230 extending to the trocar adapter 238. In another embodiment, the trocar shaft 230 includes a generally rigid conductive member covered by an insulation 244 that exposes trocar electrode 242 from the distal tip 240. The trocar shaft 230 can be constructed from stainless steel that is suitably rigid and can conduct electrical energy to the trocar electrode 242. The insulation 244 can be affixed to the trocar shaft 230 in a suitable manner such as via heat shrink or extrusion or preformed and attached to the trocar shaft 230. In some embodiments, the insulation 244 extends the entire length of the trocar shaft 230, and the trocar electrode 242 is just the distal-facing tip of the trocar shaft 230. In some embodiments, the distal tip 240 and trocar electrode 242 form a blunt or atraumatic tip so as not to pierce tissue when not energized with RF energy. (In other embodiments, the trocar distal tip can include a sharp needle to mechanically puncture tissue.)

The assembly 200 can include other features. In some embodiments, the trocar shaft 230 includes a sensor capable of sensing physiological information. The sensor can be disposed in the shaft 230 or distal tip 240 and coupled to the trocar adapter 238 for connection to components of the electrosurgical system 100. Example sensors can include an impedance sensor, temperature sensor, pressure sensor, and other sensors. For example, an impedance sensor can be used to determine whether the trocar electrode 242 has punctured a vessel wall. In connection with a dispersive electrode, the electrosurgical system 100 can determine based on a change in impedance whether the trocar electrode is disposed against the tissue or vessel wall or whether the trocar electrode 242 is disposed in the blood of a punctured vessel. Impedance readings, for instance, can help clinicians determine whether they have achieved vascular access. Additionally, the adapter portions 218, 238 of the assembly 200 can include various connections, such as a connection via cable to the RF generator, or a Luer or Luer-type connection for communication with the distal end portions 216, 234 of the assembly 200 to receive blood flow into a connected syringe. The shaft 230, distal tip 240, or both can include a radiopaque or echogenic marker such as one constructed from platinum to aid in visualization and placement.

The trocar 204 is configured to be operated with the assembly 200 for percutaneous access of a vessel. Once the trocar 204 is positioned against the skin or other tissue of the patient within the cannula 202, the distal tip 240 can be energized with the RF energy to puncture the tissue and access the vessel. The RF energy can be applied as desired continuously or in selected short bursts to puncture the skin, during tunneling through the subcutaneous tissue, and to puncture the vessel wall to access the vessel lumen. The trocar 204 can be removed from the cannula 202 and replaced with a guidewire that is inserted into the vessel. The cannula 202 can be retracted over the guidewire, and a conventional sheath in some examples is inserted over the guidewire. The trocar shaft 230 in some embodiments can also define a longitudinally extending lumen through the trocar 204 from the distal tip 230 to the adapter 238 configured to receive a guidewire to extend from the trocar 204. Once the trocar 204 has accessed the vessel, the guidewire can be inserted into the trocar 204 and into the vessel. The cannula 202 and trocar 204 can then be retracted over the guidewire.

FIG. 3 illustrates an embodiment of another vascular access assembly 300 for use with electrosurgical medical system 100 as vascular access assembly 104. The vascular access assembly 300 includes a dilator and sheath assembly 302 and an electrically active puncture member 304. In embodiments, the puncture member 304 is configured to be coupled to the RF generator 102 of system 100 and deliver a source of RF energy.

The dilator and sheath assembly 302 can include a conventional dilator 310 disposed within a sheath 312. The sheath 312 can include an elongate shaft 314 defining a longitudinally extending lumen 316 to receive the dilator 310. The sheath shaft 314 includes a proximal end 318 and a distal end 320. The dilator 310 includes a tapered distal portion 322 configured to extend from the distal end 320 of the sheath 312 with an enlargement of cross-sectional area with respect to a dilator distal tip 324. As the distal tapered portion 322 is passed through an aperture from the dilator distal tip 324, the enlargement of cross-sectional area dilates the aperture. The dilator 310 can be configured as a straight dilator, as illustrated, or a curved dilator. In one embodiment, the distal end 320 of the sheath 312 is also tapered to transition with the tapered dilator distal portion 322. The dilator 310 can include a shaft (not shown) that is disposed within the sheath 312 and that defines a lumen to receive the electrically active puncture member 304. The distal end 320 can include holes to prevent cavitation of the sheath 312 and a radiopaque or echogenic marker such as one constructed from platinum to aid in visualization. The puncture member 304 is configured to extend from the dilator distal tip 324. In some embodiments, the sheath 312 can include an inner lumen surface lined with a hydrophobic material such as polytetrafluoroethylene (PTFE) and an outer surface coated with a siloxane such as silicone. The dilator 310 can be constructed from various materials including insulative materials such as high-density polyethylene (HDPE).

The dilator and sheath assembly 302 can include a proximal handle 326 with device controls and connections configured to couple to the electrosurgical system 100. For instance, the device controls can include a releasable lock in a first configuration to grasp the puncture member 304 and hold the puncture member 304 in place with respect to the dilator distal tip 324. For example, the releasable lock can hold the puncture member as it is extended approximately 0.5 millimeters (mm) to 4.0 mm from the dilator distal tip 324. The lock in a second configuration can release the puncture member 304 and allow it to extend from the distal tip 324 or retract within the dilator and shaft assembly 302. In another example, the controls can include a slider to extend the puncture member 304 from the distal tip 324 a selected amount (such as 0.5 mm-4.0 mm) and to retract the puncture member 304 to within the dilator and shaft assembly 302. Additionally, the handle 326 can include various connections, such as a connection via cable to the RF generator, or a Luer or Luer-type connection for communication with the assembly 300 to receive blood flow into a connected syringe. The handle 326 can include a steering mechanism (not shown), such as a knob or dial, that is used to deflect or steer sheath shaft 314. In some embodiments, the handle 326 is fitted with a hemostasis valve to reduce blood loss during catheter introduction and a sideport with a stopcock to permit blood aspiration, fluid infusion, and pressure monitoring.

In the illustrated example, the puncture member 304 is configured as a multifunction conductive guidewire 304. For instance, the multifunction guidewire 304 can be used, without exchanges, as a guidewire, a vascular access puncture device, a transseptal puncture device, or as an exchange rail for delivering therapy sheaths. Such embodiments provide efficiencies to medical procedures as the puncture member 304 performs multiple functions and reduces the amount of device exchanges in the medical procedure. The multifunction guidewire 304 is sufficiently thin and flexible to access the various chambers of the heart. In some embodiments, the multifunction guidewire includes an outer diameter of between 0.25 inches to 0.038 inches. The multifunction guidewire 304 includes a distal tip 330. The distal tip 330 includes an exposed electrode 332. The multifunction guidewire 304 is configured to conduct an electrical signal, from a proximal end 334 to the exposed electrode 332. The exposed electrode 332 is configured to apply the RF energy to puncture tissue. For example, the exposed electrode 332 is electrically coupled to the electrosurgical generator 102 and is operable to deliver RF energy to puncture vasculature and tissue including derma and the atrial septum through which guidewire 304 is advanced. Once advanced through the puncture site and sufficiently extended from within the dilator distal tip 324, a distal section of the guidewire 304 is biased to form an atraumatic J-tip or coil beyond the puncture site. The sheath and dilator assembly 302 is retractable from the patient over the multifunction guidewire 304. The multifunction guidewire 304 can also support the installation of tubular members or other catheters and for advancing other devices within the heart. In some embodiments, the puncture member 304 is also implemented to perform a transseptal puncture, such as crossing member 130.

FIG. 4 illustrates an embodiment of another vascular access assembly 400 for use with electrosurgical medical system 100 as vascular access assembly 104. The vascular access assembly 400 includes an RF perforation instrument 402 and an electrically active puncture member 404. In embodiments, the RF perforation instrument 402 and puncture member 404 are configured to be coupled to the RF generator 102 of system 100 and deliver a source of RF energy.

The RF perforation instrument 402 includes an elongate instrument shaft 410 having a proximal end 412 and a distal end 414. The instrument shaft 410 defines a longitudinally extending lumen 416 that extends the length of the shaft 410. The proximal end 412 is configured to be coupled to the source of RF energy and includes an electrical coupling. The distal end 414 of the RF perforation instrument 402 includes a distal tip 420 configured to puncture tissue via RF perforation. In the illustrated embodiment of RF perforation instrument 402, the distal tip 420 includes an instrument electrode 422 to deliver RF energy to puncture tissue. For instance, the instrument electrode 422 can be electrically coupled to the RF generator via elongate conductors disposed in the instrument shaft 410 extending to the electrical coupling. In another embodiment, the instrument shaft 410 includes a generally rigid conductive member covered by an insulation 424 that exposes instrument electrode 422 from the distal tip 420. The instrument shaft 410 can be constructed from stainless steel that is suitably rigid and can conduct electrical energy to the instrument electrode 422. The insulation 424 can be affixed to the instrument shaft 410 in a suitable manner such as via heat shrink or extrusion or preformed and attached to the instrument shaft 410. In some embodiments, the insulation 424 extends the entire length of the instrument shaft 410, and the instrument electrode 422 is just the distal-facing tip of the instrument shaft 410. In some embodiments, the distal tip 420 and instrument electrode 422 form a blunt or atraumatic tip so as not to pierce tissue when not energized with RF energy. In some embodiments, the instrument shaft 410 includes a sensor capable of sensing physiological information. The sensor can be disposed in the instrument shaft 410 or distal tip 420 and coupled to the proximal end 412 for connection to components of the electrosurgical system 100. Example sensors can include an impedance sensor, temperature sensor, pressure sensor, and other sensors.

In the illustrated example, the puncture member 404 is configured as a multifunction conductive guidewire 404. For instance, the multifunction guidewire 404 can be used, without exchanges, as a guidewire, a vascular access puncture device, a transseptal puncture device, or as an exchange rail for delivering therapy sheaths. Such embodiments provide efficiencies to medical procedures as the puncture member 404 performs multiple functions and reduces the amount of device exchanges in the medical procedure. The multifunction guidewire 404 is sufficiently thin and flexible to access the various chambers of the heart. The multifunction guidewire 404 includes a distal tip 430. The distal tip 430 includes an exposed electrode 432. The multifunction guidewire 404 is configured to conduct an electrical signal, from a proximal end 434 to the exposed electrode 432. The exposed electrode 432 is configured to apply the RF energy to puncture tissue. For example, the exposed electrode 432 is electrically coupled to the electrosurgical generator 102 and is operable to deliver RF energy to puncture vasculature and tissue including derma and the atrial septum through which guidewire 404 is advanced. In some embodiments, a distal section 436 of the guidewire 404 is biased to form an atraumatic J-tip or coil beyond the puncture site. In some embodiments, the distal tip 430 and trocar electrode 432 form a blunt or atraumatic tip so as not to pierce tissue when not energized with RF energy. In some embodiments, the puncture member 404 is also implemented to perform a transseptal puncture, such as crossing member 130.

In a use of the vascular access assembly 400, the RF perforation instrument 402 is positioned against the skin or other tissue of the patient. In some embodiments, the RF perforation instrument 402 can be disposed within a cannula 406 or other delivery device. Once positioned, the instrument electrode 422 is energized with RF energy to puncture the tissue. The RF energy can be applied as desired continuously or in selected short bursts to puncture the skin, during tunneling through the subcutaneous tissue to access a vessel. The instrument distal tip 420 can be positioned against vessel wall. The multifunction guidewire 404 is inserted into the lumen 416, and the guidewire electrode 432 is positioned against the vessel wall. Once positioned, the guidewire electrode 432 is energized with RF energy to puncture the vessel wall and access the vessel lumen. The RF energy can be applied as desired continuously or in selected short bursts to puncture the vessel wall to access the vessel lumen. The RF perforation instrument 402 is retractable from the patient over the multifunction guidewire 404. The multifunction guidewire 404 can also support the installation of tubular members or other catheters and for advancing other devices within the heart and as crossing member 130 to perform transseptal puncture. The perforation instrument 402, guidewire 404, or both can include a radiopaque or echogenic marker such as one constructed from platinum to aid in visualization.

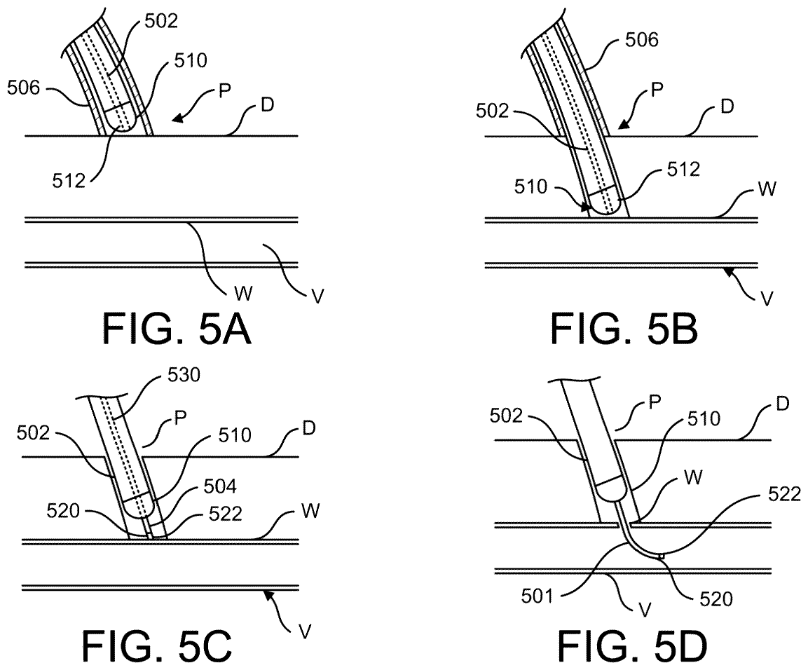

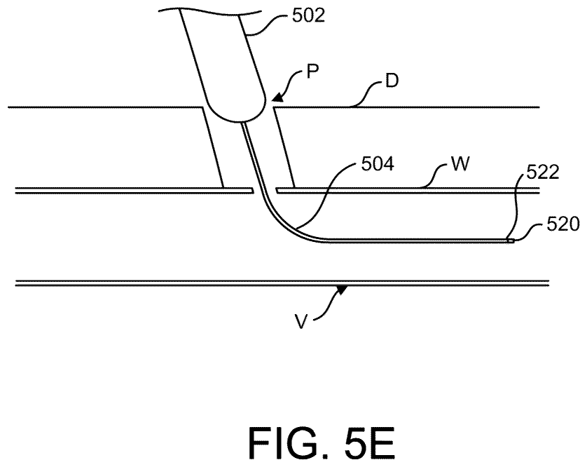

FIGS. 5A-5E illustrate a method of percutaneously accessing a vasculature in the body. For example, a vascular access assembly 104 can be used to introduce a catheter into the vasculature of a patient via an entry blood vessel V, such as a femoral artery in the patient, having a vessel wall W located beneath the derma D and tissue at puncture site S. The vascular access assembly 104 is electrically coupled to the RF generator 102 to provide a source of RF energy. In some embodiments, the method employs the use of a percutaneous instrument 502 with a distal tip 510 having an electrode 512, and the electrode 512 is coupled to the RF generator 102. The method employs the use of a puncture member 504 with a distal tip 520 having an electrode 522, and the puncture member electrode 522 is coupled to the RF generator 102. If the vascular access assembly 104 is to be configured in a monopolar mode, the patch electrode 110 is coupled to the patient. The RF generator 102 can be set to a puncture mode, or a first puncture mode, such as an energy output of approximately 10 watts. Additionally, the patient can be coupled to the visualization system 108. In some embodiments, vascular access assemblies 200 and 400 can be employed to access the method of accessing the vasculature. For instance, trocar 204 or RF perforation instrument 402 can correspond with percutaneous instrument 502, and a multifunction RF perforation guidewire 404 can correspond with puncture member 504.

FIG. 5A illustrates a distal tip 510 of the percutaneous instrument 502, such as trocar 204 or RF perforation instrument 402, is placed against the derma D of the patient at a puncture site P. In some examples, a cannula or sheath 506 is first placed against the patient to receive the percutaneous instrument 502. If the percutaneous instrument 502 includes a perforation electrode at the distal tip 510, the percutaneous instrument 502 can be pressed against the derma D without puncturing the derma D. If the percutaneous instrument 502 includes a mechanical puncturing device at the distal tip 510, such as a needle having a blade or point, an excess of a threshold pressure applied to the percutaneous instrument 502 will puncture the derma D. For a percutaneous instrument 502 with the perforation electrode 512 at the distal tip 510, a moderate amount of axial pressure is applied to the derma D and the RF energy is provided to the perforation electrode 512 to perforate the derma D tissue and other tissues, such as fat and muscle. The amount of RF energy applied in the first puncture mode can be tailored to puncture derma and tissue, such as at a first RF energy. The percutaneous instrument 502, whether via mechanical puncture or RF energy, readily advances through the tissue at the puncture site P to the vessel V, as indicated in FIG. 5B. If the percutaneous instrument 502 carries a sensor, the sensor can be used to guide the advancing percutaneous instrument 502 to the vessel wall W. In some embodiments, the percutaneous instrument 502 can be removed from the patient after the vessel wall W has been reached.

FIG. 5C illustrates the puncture member 504 is inserted into the puncture site P. In some embodiments, the puncture member 504 can be introduced into the cannula or sheath 506 after the percutaneous instrument 502 has been removed. In the illustrated embodiment, the puncture member 504 is introduced into the puncture site P via a lumen 530 extending through the percutaneous instrument 502 and the puncture member electrode 522 at the distal tip 520 is disposed against the vessel wall W. A moderate amount of axial pressure is applied to the vessel wall W and the RF energy is provided to the puncture member electrode 522 to perforate the vessel wall W and extend the puncture member distal tip 520 into the vessel lumen as illustrated in FIG. 5D. The amount of RF energy applied to puncture the vessel wall W in some embodiments is at a second puncture mode with a second RF energy that is different than the first RF energy applied to puncture the derma D and tissue. In some embodiments, the first RF energy to puncture the derma D is the same as the second RF energy to puncture the vessel wall W. The puncture member 504 can be further advanced into the vasculature, as illustrated in FIG. 5E, and the percutaneous instrument 502 and cannula or sheath 506 can be retracted over the puncture member 504 and removed from the patient. In some embodiments, a dilator/sheath assembly can be inserted into the vasculature over the proximal end of the puncture member 504 and also advanced through the vasculature.

In an anticipated use of the system 100, the puncture member 540 is a multifunction guidewire inserted into the vasculature and advanced to the superior vena cava. The dilator tip of the dilator/sheath assembly is advanced over the guidewire to the superior vena cava. Under visualization, the dilator tip is moved from the superior vena cava to the right atrial septum and then to the fossa ovalis of the heart. Once the dilator tip is confirmed at the fossa ovalis, such as via visualization, the puncture member electrode 522 is advanced from the dilator tip. In one example, the puncture member electrode 522 is extended a few millimeters from the dilator tip to tent the heart tissue and apply RF energy. In general, the puncture member 504 is extended longitudinally for several millimeters prior to the distal section curving to assume a J-tip or pigtail shape and deflecting away from the atrial septum. Forward pressure is applied to the puncture member 504 and the RF generator 102 is actuated to apply the RF energy to the puncture member electrode 522 and puncture the fossa ovalis at a puncture site in the atrial septum. The puncture member 504 can be advanced into the left atrium of the heart and anchored. The dilator tip is advanced into the puncture site of the atrial septum to expand the aperture. The instrument 502, puncture member 504, or both can include a radiopaque or echogenic marker such as one constructed from platinum to aid in visualization. While leaving a sheath in place, the dilator and puncture member 504 can be removed from the body.

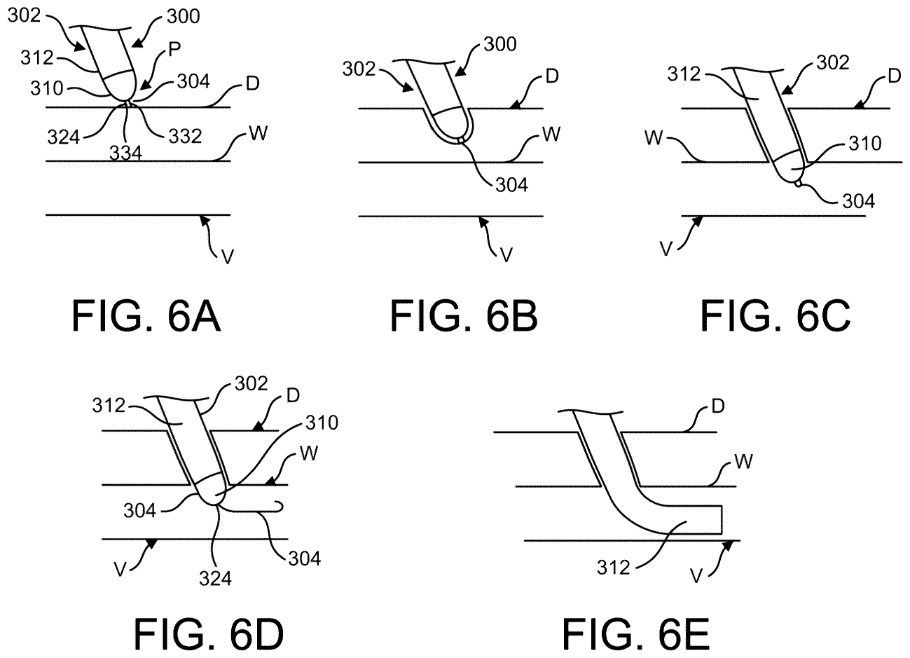

FIGS. 6A-6E illustrate a method of percutaneously access a vasculature of the body with the vascular assembly 300 including the dilator and sheath assembly 302 (having a dilator 310 and sheath 312) with the electrically active puncture member 304. For example, a vascular access assembly 300 can be used to introduce a catheter into the vasculature of a patient via an entry blood vessel V, such as a femoral artery in the patient, having a vessel wall W located beneath the derma D and tissue at puncture site S. The vascular access assembly puncture member 304 is electrically coupled to the RF generator 102 to provide a source of RF energy. If the vascular access assembly 300 is to be configured in a monopolar mode, the patch electrode 110 is coupled to the patient. The RF generator 102 can be set to a puncture mode, or a first puncture mode, such as an energy output of approximately 10 watts. Additionally, the patient can be coupled to the visualization system 108.

FIG. 6A illustrates dilator distal tip 324 is positioned near or against the derma D and the exposed electrode 334 of the conductive guidewire 304 is placed against the derma D of the patient at a puncture site P. In some examples, a cannula is first placed against the patient to receive the dilator and sheath assembly 302 with the puncture member 304. The vascular access assembly 300 can be pressed against the derma D without puncturing the derma D. A moderate amount of axial pressure is applied to the derma D and the RF energy is provided to the electrode 332 to puncture the derma D tissue and other tissues, such as fat and muscle. The amount of RF energy applied in the first puncture mode can be tailored to puncture derma and tissue, such as at a first RF energy. The derma D tissue are punctured via RF energy to create an aperture and the dilator tip pressed against the tissue opens the aperture to allow the sheath 312 to access the tissue. The vascular access assembly 300 readily advances through the tissue at the puncture site P to the vessel V, as indicated in FIG. 6B. The RF energy can be applied in continuously or in a series of bursts to permit the electrode 334 to tunnel through the tissue and the vessel wall W. If the vascular access assembly 300 carries a sensor, the sensor can be used to guide the advancing vascular access assembly to the vessel wall W. FIG. 6C illustrates the electrode 334 has punctured the vessel wall W to form an aperture and the dilator distal tip 334 has enlarged the aperture in the vessel wall to allow the sheath 312 to enter the vessel V. Once the vessel wall W has been punctured, the guidewire 304 can be advanced from the dilator distal tip 324 to form an atraumatic distal tip, such as a J-tip, as illustrated in FIG. 6D. The dilator 310 and guidewire 304 can be removed from the vessel V leaving the sheath 312 in the vasculature, as illustrated in 6E.

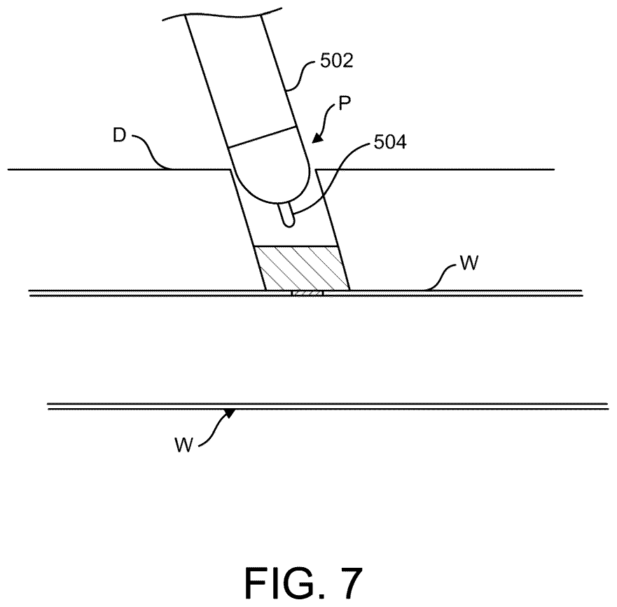

FIG. 7 illustrates the vascular access assembly 104 further being used to help close the puncture site P. For example, vascular access assemblies 200, 300, 400 having be applied to puncture derma D at the puncture site P and puncture the vessel wall W access at site S, such as via one or more puncture settings on RF generator 102. An RF electrode, such as via percutaneous instrument 502 or puncture member 504, which in some embodiments includes trocar 204, puncture member 304, RF perforation instrument 402, or puncture member 404, is set to a cauterization setting. The cauterization setting applies an RF energy that is different than the RF energies used to puncture either the derma D, the vessel wall W, or the atrial septum. The vascular access assembly 104 is applied to cauterize and aid to close the puncture site P.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method of percutaneously accessing vasculature in a body, the method comprising:
advancing, against tissue in the body, a percutaneous instrument having an elongate shaft defining a longitudinal lumen and an instrument distal tip, the instrument distal tip configured to puncture the tissue at a puncture site; and
advancing, against a vessel in the body accessed from the punctured tissue, an RF perforation guidewire extendable from the lumen, the RF perforation guidewire having an atraumatic guidewire distal tip, the guidewire distal tip having a guidewire electrode delivering RF energy to puncture the vessel at the puncture site.

2. The method of claim 1, wherein the instrument distal tip includes a needle mechanically puncturing the tissue at the puncture site.

3. The method of claim 1, wherein the instrument distal tip includes an instrument electrode configured to deliver RF energy to puncture the tissue at the puncture site.

4. The method of claim 3, wherein delivering the RF energy to puncture the tissue and delivering the RF energy to puncture the vessel include delivering the RF energy in a monopolar mode.

5. The method of claim 1, wherein the RF perforation guidewire is introduced into the lumen after the vessel is punctured.

6. The method of claim 1, wherein the guidewire distal tip is extendable from the instrument distal tip such that the percutaneous instrument is fully retractable over the RF perforation guidewire.

7. The method of claim 1, further comprising advancing, into a left atrium of a heart of the body, the Rf perforation guidewire via the vessel from the puncture site.

8. The method of claim 7, further comprising puncturing an atrial septum of the heart with the atraumatic guidewire distal tip having the guidewire electrode delivering RF energy.

9. The method of claim 1, further comprising cauterizing the puncture site.

10. A method of percutaneously accessing vasculature in a body, the method comprising:
puncturing tissue at a puncture site in the body;
puncturing a vessel in the body accessed from the punctured tissue, the puncturing the vessel with an electrode delivering a first RF energy at the puncture site; and
cauterizing the puncture site with the electrode delivering a second RF energy, wherein the first RF energy is different from the second RF energy.

11. The method of claim 7, wherein the puncturing the tissue includes puncture the tissue with the electrode delivering a third RF energy at the puncture site.

12. The method of claim 11, wherein the first RF energy is the same as the third RF energy.

13. A percutaneous vascular access assembly for use with a radiofrequency (RF) energy source, the percutaneous vascular access assembly comprising:
an RF perforation instrument configured to be coupled to the RF energy source, the RF perforation instrument having an elongate shaft defining a longitudinally extending lumen and an instrument distal tip having an instrument electrode configured to deliver the RF energy; and
a puncture member adapted to be disposed within the lumen and extendable from the instrument distal tip and configured to be coupled to the RF energy source, the puncture member having a puncture member distal tip having a puncture member electrode configured to deliver the RF energy.

14. The percutaneous vascular access assembly of claim 13, and further comprising a cannula configured to receive the RF perforation instrument.

15. The percutaneous vascular access assembly of claim 13, wherein the RF perforation instrument is configured as a trocar.

16. The percutaneous vascular access assembly of claim 13, wherein the RF perforation instrument includes a sensor.

17. The percutaneous vascular access assembly of claim 13, wherein the puncture member is configured as a multifunction guidewire.

18. The percutaneous vascular access assembly of claim 17, wherein the guidewire includes an atraumatic distal section.

19. The percutaneous vascular access assembly of claim 13, and further comprising an RF generator to provide the RF energy source.

20. The percutaneous vascular access assembly of claim 13, wherein the RF perforation instrument and the puncture member are configured to deliver the RF energy in a monopolar mode.
